SEQUENCE LISTING (1 sequence)

SEQ ID NO: 1 - DNA, 1753 bp, Artificial Sequence
  Synthetic construct
taatacgactcactatagggaaataagagagaaaagaagagtaagaagaaatataagagccaccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgcagggaggcgtcatgggtggcagcaactccaaccagcagcaacagatgcgactgcagcaactgcagatggagaaggagaggctgcggctgaaacagcaagaactgcttcggcaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttataggctgccttctgcggggcttgccttctggccatgcccttcttctctcccttgcacctgtacctcttggtctttgaataaagcctgagtaggaag

(19) United States
(12) Patent Application Publication
(10) Pub. No.: US 2019/0136200 A1
(43) Pub. Date: May 9, 2019

(54) Cellular Reprogramming Utilizing mRNA

(71) Applicant: Luigi WARRE, San Diego, CA (US)

(72) Inventor: Luigi WARRE, San Diego, CA (US)

(21) Appl. No.: 16/068,469

(22) PCT Filed: Dec. 29, 2016
(86) PCT No.: PCT/US2016/069079

§ 371 Date: Jul. 9, 2018

Related U.S. Application Data
(60) Provisional application No. 62/276,520, filed on Jan. 8, 2016.

(51) Int. Cl. C12N 5/074; C07K 14/47; A61K 35/545

(52) U.S. Cl. CPC C12N 5/0696; C07K 14/4702; A61K 35/545; C12N 2506/1307; C12N 2510/00; C12N 2501/603; C12N 2506/11; C12N 2501/605; C12N 2506/45; C12N 2506/094; C12N 2506/1384; C12N 2506/28; C12N 2501/602

(57) ABSTRACT

Methods for accelerated cell lineage conversion and the treatment of patients with the lineage converted cells are provided. The methods include the steps of transfecting a cell with a composition that includes at least one synthetic mRNA encoding a chimeric protein that corresponds to an engineered fusion of a transcription factor and an heterologous peptide sequence derived from the C-terminal TAD of Gal4. The TAD domain enhances the epigenetic remodeling activity of the chimeric protein increasing the speed of lineage conversion. The converted cells may be used for research or administered to a human or animal patient as a therapy. In one preferred embodiment, the reprogramming of a somatic cell to pluripotency is accelerated by using a cocktail of mRNAs expressing a combination of wild-type or engineered reprogramming factors where Oct4 and/or Sox2 and/or Nanog are expressed as Gal4 TAD chimeras.

### CROSS-REFERENCE TO RELATED APPLICATIONS

Not applicable

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION BY REFERENCE OF MATERIAL SUBMITTED ON A COMPACT DISC

Not applicable

### TECHNICAL FIELD

The present invention relates generally to the field of molecular biology and the reprogramming of cells to convert them from one specialized phenotype to another. More specifically, it relates to the use of synthetic mRNAs encoding chimeric transcription factors incorporating a transactivation domain from the carboxy-terminus of the Gal4 transcription factor of Saccharomyces cerevisiae to promote accelerated lineage conversions in human and animal cells.

### BACKGROUND OF THE INVENTION

### Utilizing Transgenes to Manipulate Cell Fate

Researchers have understood since the 1980s that ectopic gene expression techniques can be used to manipulate cell lineage in a dish, converting cells from one specialized phenotype to another. An early demonstration of this idea was an experiment showing that fibroblasts can be converted into cells displaying the characteristic features of muscle cells upon transfection with a synthetic plasmid construct expressing MyoD, a key regulator of myogenic development in vivo. This represents an engineered “transdifferentiation,” (i.e., a direct conversion of a somatic cell from one terminally-differentiated cell type to another). The genes which can be used to promote such lineage conversions are typically “transcription factors,” (i.e., they belong to the class of proteins, which interacts directly with DNA in a sequence-specific manner to regulate the expression of other genes). In some cases, genes encoding other types of proteins or certain non-coding RNAs such as microRNAs and long non-coding RNAs can also affect cell fate. Importantly, cell lineage conversion does not require indefinite transgene expression because the various naturally-occurring cell types represent stable “attractors” in gene expression space: once established, their underlying pattern of gene expression is self-reinforcing and refractory to ordinary perturbations. Characteristically, the ectopic expression of regulatory factors governing cell lineage has to be sustained for at least several days to activate a stable pattern of genetic regulatory network activity and remodel the epigenetic state of the chromatin sufficiently to effect a lasting change in cellular phenotype.

### Induced Pluripotent Stem Cells

The level of interest in artificially-induced cell lineage conversion has surged in recent years, largely in response to the breakthrough demonstration that the co-expression of a handful of transcription factors can dedifferentiate somatic cells such as fibroblasts to a primitive, uncommitted state closely resembling that of the “embryonic stem cells” (ESCs) which have been isolated from early-stage embryos. The term “cellular reprogramming” is often used for this induced dedifferentiation process. Like ESCs, “induced pluripotent stem cells” (iPSCs) are immortal, i.e., they can be expanded indefinitely in a dish, and in principle they can be coaxed by a process of “directed differentiation” to give rise to somatic cells of any desired type (e.g., dopaminergic neurons, cardiac progenitors, retinal epithelial cells and pancreatic beta cells). The import of this work was recognized by the award of a Nobel Prize to Shinya Yamanaka in 2012. Yamanaka was the first to demonstrate iPSCs and the term “Yamanaka factors” is often used to refer to a set of four transcription factors (i.e., Oct4, Sox2, Klf4 and c-Myc), which emerged from his complex screening experiments as a minimal combination that can reprogram fibroblasts to iPSCs with useful efficiency.

The interest in iPSCs reflects powerful benefits of this technology. These pluripotent cells can be used to derive specialized somatic cells that cannot be readily established as primary cultures, for example specific dopaminergic neuronal subtypes that could be used to investigate the biology of Parkinson's Disease and to screen or evaluate possible treatments in a dish. Because iPSCs can be derived from an adult patient biopsy, they sidestep the ethical concerns and regulatory issues that have impeded the exploitation of ESCs. In contrast to ESCs, these pluripotent cells can also be made in limitless variety to represent different genetic endowments including hereditary diseases. Potentially, iPSCs could be used to make cells, tissues or organs for transplant back to the original somatic cell patient donor (so-called “autologous transplantation”), minimizing or eliminating rejection and the need for immunosuppressive drugs. Human trials have already commenced using iPSC-derived retinal cells for the treatment of macular degeneration, and earlier-stage studies addressing a wide variety of clinical applications in regenerative medicine and tissue-replacement therapy are ongoing.

### Other Applications of Lineage Conversion

While reprogramming to pluripotency has generated the greatest interest, other forms of artificially-induced cell lineage conversion are currently under investigation. Relatively few fate switches can be accomplished by the expression of a single factor (as in MyoD example), but recently multi-factor cocktails comprising transcription factors and/or microRNAs have been identified which promote useful lineage conversions (e.g., from easily-obtained fibroblasts to neuronal cell types). The idea of using transgenes to fine-tune the fate of stem cells or progenitors is also garnering more attention, even if this approach is still relatively unexplored compared to traditional methods of directed differentiation based on the application of extracellular growth factors and small molecules. For example, a great deal is known about the transcription factors which specify the “A9” midbrain dopaminergic neurons involved in Parkinsonism, and the literature reports efforts to channel developing neural progenitors to this fate by ectopically expressing various combinations of these factors in cell culture.

### Disadvantages of Virus-Based Conversion Methods

In the early experiments on fibroblast-myogenic conversion mentioned above, MyoD was expressed from a plasmid (i.e., a circular piece of DNA that survives temporarily in the cell nucleus following transfection and is subsequently lost or diluted out during cell division). By contrast, subsequent work in the field of lineage reprogramming has relied heavily on the use of integrating viral vectors, in which transgene expression cassettes are packaged into viruses that copy their genome into cellular DNA as part of their natural life cycle. These viral techniques facilitate the task of expressing lineage-regulating factors robustly for the time required to effect stable fate conversion and are particularly useful when multiple factors must be co-expressed and/or the target cells undergo rounds of cell divisions over the course of the conversion. The induction of pluripotency represents the “Mount Everest” of lineage conversion as it involves pushing the state of a fully differentiated cell all the way back to a primitive, embryonic pattern of gene expression. It can take weeks of expression of the four-factor Yamanaka cocktail to induce a stable conversion in human fibroblasts. Even so, the efficiency of the process is typically very low with well under 1% of the fibroblasts giving rise to iPSC colonies. Yamanaka's work relied on the use of integrating viral vectors to meet this technical challenge, and this remains the most popular approach to making iPSCs in labs across the world today.

There are major drawbacks to the use of integrating viral vectors to make iPSCs. In the first place, the level and quality of temporal control over gene expression afforded with these vectors is limited as (a) expression cassettes generally integrate at random chromosomal locations and their activity is subsequently influenced by genomic context, and (b) endogenous genomic defense mechanisms tend to silence integrated cassettes with variable kinetics and finality. It has been reported that “leaky” expression or unintended reactivation of integrated reprogramming factor cassettes leads to problems with the reproducibility of directed differentiation performed on iPSCs made by viral methods, compromising their utility even for purely research-oriented applications such as drug discovery. Of still greater concern, any reprogramming method that leaves copies of oncogenes such as c-Myc embedded at random locations in the genome is unlikely to receive approval in regenerative medicine applications owing to the risk that these cassettes might become reactivated in a patient and cause cancer.

A consensus quickly emerged within the iPSC research community that the development of so-called “footprint-free” reprogramming techniques to avoid the problem of genomic alteration would be of key importance to realizing the promise of these cells. Several alternative technologies to address this need have been reported, and already some of these methods have seen significant levels of adoption by workers in the field.

### Footprint-Free Reprogramming Methods

The reprogramming methods that have been developed to avoid the problem of genomic integration can be grouped into three classes:

Class A—Techniques based on “excisable” integrating vectors. In one popular approach, the use of lentiviral vectors featuring flanking recombination sites allows integrated transgenes to be edited out through a post-reprogramming cleanup step based on brief expression of a recombinase enzyme by transient transfection of a plasmid or messenger RNA. Another approach uses a transposon vector to embed transgene expression cassettes in the genome. After reprogramming, plasmid or mRNA transfection can be used to express a transposase enzyme to purge integrated transposon sequences from the genome.

Class B—Techniques based on non-integrating DNA vectors. Common variations involve the use of multiple rounds of plasmid transfection or, alternatively, one-shot transfection of an “episome” (i.e., a circular DNA featuring a eukaryotic origin of replication included to prolong transgene expression in dividing cells). Reprogramming has also been reported using non-integrating adenoviral vectors, although this method has not seen wide adoption.

Class C—Techniques based on non-DNA expression vectors such as protein or RNA molecules, or viruses having completely RNA-based life cycles. This class include delivery of reprogramming factors in the form of recombinant proteins featuring cell membrane-penetrating peptide domains (referred to as “protein transduction”), transfection with synthetic mRNA or microRNA (or some combination of both), transfection of special self-replicating mRNA molecules that exploit features derived from alphaviruses, and the use of Sendai virus as an expression vector.

While the techniques of Class A and B can be applied to generate footprint-free iPSCs, they nevertheless entail a significant risk of genomic alteration owing to incomplete excision or stochastic recombination events involving the DNA vector. In clinical applications, comprehensive screening to detect such problems would presumably be required to qualify the iPSC lines before use. While excisable lentivirus and episomal DNA vectors are currently popular technologies due to their ease of use, it seems doubtful that they will become long-term methods of choice for clinical iPSC derivation given the availability of alternative techniques that sidestep the genomic alteration problem entirely.

Of the “footprint-free” methods of Class C, protein transduction, the first to be published, has so far proved too inefficient to gain wide adoption. By contrast, Sendai virus-based reprogramming has achieved considerable popularity owing to its relatively high efficiency and “one-shot” simplicity. However, this technique does entail the use of a virus that can take weeks to clear from the resultant iPSC colonies, and again screening (with some attendant risk of false negative results) would be required before Sendai-derived iPSCs could be qualified for clinical use. Although not currently as popular as Sendai, the mRNA reprogramming system has been taken up by numerous labs despite the handicap of being fairly labor-intensive since the short-lived RNA transcripts must be redelivered daily over the course of reprogramming. Importantly, the mRNA approach avoids the cleanup/screening problem completely. MicroRNA has so far shown more utility as an adjunct to mRNA in reprogramming rather than as a standalone system. Reprogramming with self-replicating mRNA is a new approach that offers the “single-shot” convenience of Sendai but, as with the RNA virus, the relatively poor control afforded over the reprogramming factor expression time course and the potential persistence of self-replicating vector may be of concern in a clinical context.

Drawbacks of mRNA Reprogramming

In view of the foregoing discussion, it seems likely that mRNA reprogramming will ultimately emerge as the technology best-suited to bringing iPSCs to the clinic. As mRNA is rapidly degraded in living cells and is not a substrate for genomic recombination, this technology obviates any need to screen for residual traces of vector after reprogramming (whether in the form of genomic lesions, live virus, or self-replicating molecules in the cytoplasm) and eliminates vector persistence as a safety concern. It affords remarkably precise, multi-factorial control over transgene expression for reprogramming and other cell-lineage conversion applications. For reasons that are not well understood, mRNA reprogramming in human fibroblasts also tends to be significantly faster and (at least when applied to high-quality, low-passage cells) more efficient than other reprogramming systems. Reprogramming using mRNA has also been reported to be associated with a greatly reduced burden of chromosomal abnormalities when compared to several popular alternative methods.

As currently practiced, mRNA reprogramming has certain drawbacks which have slowed its rate of adoption compared to Sendai and episomal reprogramming:

(1) As mRNA transcripts have a half-life on the order of 24 hours in the cytoplasm, reprogramming cultures must be transfected on a consistent daily schedule to obtain robust outcomes. The first successful mRNA reprogramming protocols called for at least two weeks' of daily transfection to generate iPSCs. Clearly, the convenience of one-shot reprogramming systems based on viruses, episomal DNA or self-replicating mRNA outweighs the benefits of the mRNA system for many prospective users. Aside from the hands-on time involved, the need to perform a long series of transfections when using the mRNA system adds to the cost of the materials required, including the synthetic mRNA, transfection reagent, and the costly B18R protein commonly used as a media supplement to inhibit host innate immune responses to RNA.

(2) Compared to systems based on “one-shot” vectors, it has so far proved relatively difficult to translate the success of mRNA reprogramming in human fibroblasts to other cell types. Although fibroblasts remain the most popular starting material for iPSC generation, there is great interest in performing reprogramming on blood-derived cell types in particular. A central difficulty in adapting the mRNA reprogramming system to blood-derived cells is the low efficiency of transfection attainable with popular cationic transfection reagents. By contrast, transfection efficiencies of >50% are readily achieved in fibroblasts. Schematically, one can imagine that if just 10% of blood cells take up a significant amount of nucleic acid on transfection that could still support acceptable levels of reprogramming in the case of a persistent integrating or self-replicating vector. However, only a very small percentage of cells will undergo sustained, robust reprogramming factor expression over a course of repeated mRNA transfections. Electroporation is an alternative modality which can transfect RNA efficiently into blood cells. However, a prolonged regimen of daily electroporation might well prove too harsh on target cells to be useful for reprogramming.

It will be apparent from the foregoing that technical improvements that accelerate reprogramming represent a fruitful avenue for addressing the current limitations of the mRNA method. Several approaches which might speed up the process have been proposed, including (a) use of alternative combinations or optimized stoichiometries of naturally-occurring reprogramming factors; (b) use of engineered transcription factors featuring novel or chimeric peptide domains that potentiate their reprogramming effect; and (c) augmentation of the mRNA cocktail with select microRNAs or small-molecule compounds.

Early work showed that the addition of a fifth factor, Lin28, to the canonical 4-factor Yamanaka cocktail noticeably improved the speed and efficiency of mRNA reprogramming. Subsequently, the number of days of mRNA transfection required to achieve efficient fibroblast reprogramming has been cut substantially (down to 6-12 days) compared to early protocols through the addition of a sixth factor, Nanog, combined with use of either (a) an mRNA encoding an engineered variant of Oct4 (designated “M₃O”) incorporating a powerful extra transactivation domain excerpted from the MyoD transcription factor or (b) the transfection of synthetic microRNA analogs as a “boost” along with mRNA transfections. Importantly, the resulting abbreviated transfection regimens support convenient and clinically-relevant protocols that obviate the need for a feeder-cell support layer or a mid-reprogramming passaging step.

In spite of these advances there remains a pressing need to further speed up the process so that the transfection regimen can be executed within the span of the normal work week, and to facilitate the development of mRNA reprogramming protocols applicable to alternative somatic cell types.

The forgoing examples of related art and limitation related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

The present invention provides methods and compositions for accelerated cell lineage conversion. The method includes the steps of transfecting a cell with a composition that includes at least one mRNA encoding an engineered, chimeric transcription factor having a heterologous peptide sequence derived from the acidic transactivation domain (TAD) found in the C-terminal region of the yeast transcription factor Gal4. The presence of the TAD enhances the activity of the engineered chimeric transcription factor(s), resulting in substantially faster and/or more efficient lineage conversion. The lineage conversion promoted by the mRNA can be a dedifferentiation, a transdifferentiation (“direct conversion”), or a directed differentiation.

In one embodiment of the present invention the cell lineage conversion may be a dedifferentiation that reprograms the cell, generally a somatic cell, into an induced pluripotent stem cell. The starting cell subjected to reprogramming may be (but is not limited to) one of the following cell types: fibroblasts, renal epithelial cells, keratinocytes, adipose-derived stem cells, mesenchymal stem cells, blood-derived endothelial progenitors and/or peripheral blood mononuclear cells. In addition, the starting cell may be either human or non-human.

In one embodiment, the composition comprises a cocktail of at least four different mRNA species encoding reprogramming factors selected from the list Oct4, Sox2, Klf4, Myc, Lin28 and Nanog, and which includes one or more Gal4 TAD fusion constructs based on factors selected from the group Oct4, Sox2 and Nanog.

Another aspect of the present invention is a therapeutic method comprising the steps of isolating somatic cells from a patient, transfecting the somatic cells with a composition comprising at least one mRNA encoding a chimeric transcription factor having a heterologous peptide sequence derived from the C-terminal TAD of Gal4, wherein the activity of the chimeric transcription factor is enhanced by the presence of said transactivation domain; and administering the transfected cells into the patient. The somatic cells may be native unmodified cells or they may be cells that may have been genetically modified (e.g., cells in which an undesired genetic mutation like sickle cell anemia has been corrected). The method of reprogramming may be dedifferentiation, transdifferentiation or directed differentiation. The transfected cells may be administered immediately following transfection or after they are reprogrammed. The cells may be administered to the patient after being differentiated in vitro and/or being genetically modified (e.g., to correct a genetic disease). The somatic cells may be human or non-human cells and the patient being treated may be human or non-human.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects, features, and advantages of the invention will be brought out in the following part of the specification, wherein detailed description is for the purpose of fully disclosing the invention without placing limitations thereon.

### BRIEF DESCRIPTION OF THE DRAWINGS

FIG. 1 shows an example of one mRNA of the present invention containing a T7 RNA polymerase promoter, an unstructured 5′ UTR leader sequence, a strong Kozak sequence, a human Oct4 protein coding sequence fused via a linker to a Gal4 TAD sequence, and a 3′ UTR sequence excerpted from a mouse alpha-globin mRNA transcript. A: T7 RNA polymerase promoter (green); B: Unstructured 5′ UTR leader sequence (yellow); C: Strong Kozak sequence (red); D: Human Oct4 protein coding sequence (includes start codon, gray): E: Linker coding sequence (blue) F: Gal4 C-terminal TAD coding sequence (includes stop codon, fushia) and G: Murine alpha globin 3′UTR (brown).

FIG. 2 shows phase contrast and immunostaining imagery of iPSCs derived from human dermal fibroblasts by mRNA reprogramming using a six-factor cocktail comprising the Oct4-Gal4 TAD fusion construct given in FIG. 1 along with Sy, Klf4, c-Myc T58A, Lin28 and Nanog, confirming they display typical pluripotent stem cell morphology and express canonical pluripotency markers. A: 10× phase contrast image of emergent (passage 0) iPSC colonies at day 11 of reprogramming with a six-factor mRNA cocktail incorporating the Oct4-Gal4 TAD construct. B: 10× image of DAPI-stained passage 2 iPSCs derived from the reprogramming well shown in panel A at day 14, revealing the cell nuclei. C: OCT4 immunostaining of the culture shown in panel B, demonstrating the appropriately nuclear-localized expression of this canonical pluripotent stem cell marker. D: TRA-1-60 immunostaining of the cells shown in panels B and C, confirming generalized expression of this canonical pluripotency marker.

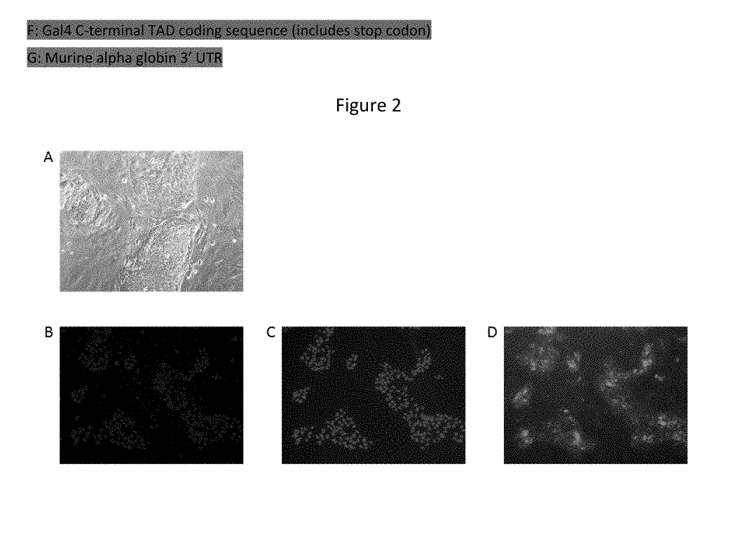

### DETAILED DESCRIPTION OF THE INVENTION

### Definitions

The term “cell lineage” as used herein refers to a cell's position within a hierarchically-organized tree of phenotypic specialization such as unfolds over the course of development in almost all multicellular organisms.

The terms “differentiation,” “differentiating” and “differentiated” as used herein refer to the developmental process by which cells take on more specialized phenotypes or give rise to more specialized progeny.

The term “lineage potential” as used herein refers to the range of possible lineages open to a cell or its clonal progeny. For example, the pluripotent cells in the inner cell mass of the early embryo have the potential to give rise to all somatic lineages, the hematopoietic stem cells (“HSCs”) found in the bone marrow of an adult mammal have the potential to give rise to the myeloid, erythroid and lymphoid lineages, and HSC-derived lymphoid progenitors can give rise to the yet more-restricted B and T cell lineages.

The term “dedifferentiation,” “dedifferentiated” and “dedifferentiating” as used herein refer to processes (typically artificially induced) by which a cell or its progeny become less specialized in phenotype and broader in lineage potential.

The terms “messenger RNA” and “mRNA” as used herein refer to an RNA molecule that is competent to be translated into a specific, encoded polypeptide by the ribosomes and associated machinery present in living cells.

The term “microRNA” and “miRNA” as used herein refers to a class of naturally-occurring small, non-coding RNA transcripts that interact with cognate mRNAs based on sequence complementarity and generally seem to regulate or silence their targets through effects on translation and turnover. These terms are also used for synthetic analogs of these transcripts.

The term “transgene” as used herein refers to a nucleic acid or polypeptide corresponding to a gene or gene product that is expressed inside cells in culture or in vivo by means of an artificial vector such as an engineered virus, transposon, plasmid, artificial mRNA, miRNA analog or cell-penetrating peptide.

The term “ectopic expression” as used herein refers to the expression of a gene or gene product in cells outside the context in which it is normally expressed (e.g., owing to the delivery of an artificial transgene, or naturally as the consequence of a mutation affecting gene regulation in cancer).

The terms “self-renew” and “self-renewal” as used herein refer to cell divisions in which at least one daughter cell shares the phenotype and lineage potential of the parental cell.

The term “stem cell” as used herein refers to a partially or completely undifferentiated cell having both the capacity to self-renew and the ability to give rise to more specialized daughter cells. This includes the cells of the early embryo and certain cells in the adult organism that serve to replenish the body's stock of differentiated cells.

The term “pluripotent stem cell” or “PSC” as used herein refer to a stem cell with the potential to give rise to specialized progeny from the three foundational lineages that emerge at the beginning of development in animals (mesoderm, ectoderm and endoderm). Such cells may be isolated from the early embryo (“embryonic stem cells” or “ESCs”) or induced artificially from differentiated cells (“induced pluripotent stem cells” or “iPSCs”). The ability of pluripotent stem cells to develop into the three foundational lineages distinguishes them from the more limited oligopotent or multipotent adult stem cells which replenish the stocks of specific lineages in the mature organism (e.g., hematopoietic stem cells).

The term “progenitor” as used herein refers to a partially-differentiated cell that has little or no capacity for self-renewal, but which has the potential to give rise to specialized cells of at least one lineage. Such cells arise as intermediates in the process of cellular differentiation.

The term “terminal differentiation” as used herein refers to a differentiation process which yields a fully specialized (i.e., “terminally differentiated”) cell that is incapable of further differentiation in the course of normal development. Terminally differentiated cells can sometimes be induced to dedifferentiate by artificial means, e.g., by cellular reprogramming.

The term “lineage commitment” as used herein refers to a decision effected within the cell at the level of the gene regulatory network to take on a specific differentiated phenotype. The maturation of this phenotype may take some time and/or be realized only within the clonal descendants of the committed cell.

The term “chromatin” as used herein refers to the complexes of DNA wrapped around histone proteins that make up eukaryotic chromosomes, the local and long-range structure of such complexes being associated with the regulation of gene expression and consequently cellular phenotype.

The terms “epigenetic,” “epigenetics” and “epigenome” as used herein refer to the status of a gene, set of genes or an entire genome apart from the aspect of heritable DNA sequence content, particularly with regard to the levels of transcriptional activity and/or the condition of the chromatin at loci of interest. The term “epigenetic change” is often used to refer specifically to phenomena which influence transcriptional activity by altering the local conformation of the chromatin to make loci more or less susceptible to transcription, often involving covalent chemical changes such as DNA or histone methylation.

The term “transcription factor” as used herein refers to a protein that modulates the activity of one or more target genes, typically by binding specific DNA sequences close to the genes and then either directly interacting with the machinery of transcription (e.g., RNA polymerase and/or various accessory proteins) or indirectly affecting the recruitment of this machinery through changes to the local chromatin architecture.

The term “DNA-binding domain” as used herein refers to an amino acid sequence within a protein (e.g., a transcription factor) that mediates sequence-specific non-covalent binding of the protein to DNA.

The term “transactivation domain” or “TAD” as used herein refers to an amino acid sequence within a transcription factor that mediates the factor's effects on transcription (e.g., by promoting or inhibiting the recruitment of RNA polymerase and/or associated proteins, or through covalent changes to the DNA or histones that alter the accessibility of the DNA to the transcriptional apparatus).

The term “transdifferentiated”, “transdifferentiating” and “transdifferentiation” as used herein refers to a process by which a differentiated cell of one specialized type is converted into a cell of different type without going through a stem cell-like intermediate state, such as when a fibroblast is directly converted into a neuron. Such transdifferentiations can be artificially induced (e.g., through expression of lineage-specific transcription factors or miRNAs). While there have been reports in the scientific literature that similar conversions occur spontaneously in vivo, these findings have not been widely accepted.

The term “direct conversion” as used herein is synonymous with “transdifferentiation”.

The term “directed differentiation” as used herein refers to the guided differentiation of a stem or progenitor cell to a specific lineage fate (e.g., through the use of specific cytokines or small molecules in culture media or by the expression of lineage-specific transcription factors or miRNAs from transgenes).

The term “somatic cell” as used herein refers to a cell contributing to the fully-formed body of a multicellular organism outside of the germ line (also referred to as sex cells) and distinguished from the undifferentiated stem cells making up the early embryo.

The terms “reprogram,” “reprogrammed,” and “reprogramming” as used herein refer to the process by which a differentiated somatic cell is dedifferentiated into a pluripotent stem cell based on ectopic expression of reprogramming factors from transgene vectors, and more broadly to technologically-induced cell lineage conversion in general.

The term “reprogramming factor” as used herein refers to a transgene utilized to promote cellular reprogramming, often (but not necessarily) a transcription factor or microRNA.

In the context of cell fate manipulation, the term “cocktail” as used herein refers to a combination of two or more reprogramming factors used in conjunction to promote lineage conversion.

The term “transfect,” “transfects,” “transfecting” and “transfection” as used herein refer to the delivery of nucleic acids (usually DNA or RNA) to the cytoplasm or nucleus of cells (e.g., through the use of a cationic lipid vehicle or by means of electroporation).

The term “modified base” as used herein refers to a chemically-distinct variation on one of the canonical nucleobases (i.e., adenosine (DNA/RNA), cytosine (DNA/RNA), guanine (DNA/RNA), thymine (DNA) and uridine (RNA)). The chemical modification may take the form of isomerism (as in the case of the uridine variant pseudouridine) or the presence of a “decorating” chemical group (as in the case of the cytidine variant 5-methylcytidine).

The term “modified nucleotide” as used herein refers to a nucleotide triphosphate featuring a modified base, sugar or backbone moiety.

The term “heterologous peptide” as used herein refers to an amino acid sequence engineered into a modified version of a naturally-occurring protein, the sequence typically corresponding to a functional domain excerpted from another naturally-occurring protein and usually endowing it with greater potency or novel functionality.

The term “fusion protein” as used herein refers to an engineered polypeptide that combines sequence elements excerpted from two or more naturally-occurring proteins.

The term “chimeric transcription factor” as used herein refers to an artificial transcription factor engineered by combining components excerpted from two or more naturally-occurring proteins together in a fusion construct.

The term “enhanced activity”, in the context of engineered transcription factors, refers to alterations to a native protein sequence that exaggerate the factor's effects on transcriptional activity at target genes (e.g., by increasing the degree to which the factor promotes or inhibits recruitment of transcriptional machinery such as RNA polymerase and/or its accessory proteins, or by increasing the rate of covalent changes to local chromatin where these changes mediate the factor's effects on transcription).

The term “Gal4” as used herein refers to a transcription factor expressed in the yeast species Saccharomyces cerevisiae.

Described herein are detailed compositions and methods for changing the lineage of human or animal cells by means of synthetic mRNAs expressing chimeric versions of transcription factors which have been potentiated through the incorporation of a TAD peptide sequence excerpted from the C-terminal region of Gal4. The methods and compositions described herein are faster than currently known methods, avoid the cleanup and screening requirements and residual risks associated with the use of DNA-based gene expression vectors, all of which undergo recombination with cellular genomes (either necessarily by their mode of action or stochastically at low frequency) including retroviral, lentiviral, adenoviral, transposon, plasmid and episomal vectors. The methods and compositions described herein are also easier to control and do not suffer from the cleanup and screening requirements and residual risks associated with expression vectors based on RNA viruses (e.g., Sendai virus) or self-replicating mRNA molecules. The accelerated lineage conversions facilitated by the methods and compositions described herein can reduce costs and turnaround times, relax burdensome technical constraints such as the need to grow target cells on feeder cells (which is inconvenient and also problematic for clinical applications), and lower the need to employ costly countermeasures to abrogate cellular immune responses to the administration of exogenous RNA. Further, by significantly reducing the number of transfections needed to achieve lineage conversion through faster remodeling of the epigenome, the methods and compositions described herein facilitate application of clinically-relevant mRNA-based methods to cell types such as blood cells which, are otherwise refractory to this general approach owing to the difficulty of achieving efficient, sustained delivery of nucleic acids to the cells in culture.

### Cellular Differentiation and Lineage Conversion

Multicellular organisms (“metazoans”) are made up of complex communities of cells expressing diverse phenotypes. For example, it is estimated that there are two hundred distinct cell types in the human body. This diversity of cellular phenotypes reflects the adaptive advantages, which proceed from realizing a division of labor in the organismal context. The various cellular phenotypes are manifestations of distinct gene expression profiles, with the expression profile at the level of gene transcription determining the contents of the proteome, which in turn dictates cellular structure and function. For instance, liver cells uniquely express certain specialized enzymes involved in degrading toxic metabolic waste products, while neurons distinctively express specialized cytoskeletal proteins that support the extension of processes required for long-range cell-cell signaling. It is currently understood that since every metazoan cell community arises from a single, founding zygote, the phenotypically diverse cells found in the mature organism emerge clonally from less specialized and ultimately completely unspecialized ancestor cells. The process of phenotypic diversification and specialization is referred to as “cellular differentiation.” Cellular differentiation proceeds in a hierarchical fashion, with the growing cellular community progressively partitioned into subpopulations with increasingly specialized characteristics and more restricted fate potential. The differentiation status of a cell is often referred to as its “lineage” in recognition of the nested, branching character of this process. The first stage in the differentiation process is called “gastrulation” and occurs when the superficially homogenous ball of cells that comprises the early embryo segregates into three distinct “germ layers” designated the “mesoderm,” “endoderm” and “ectoderm.” These three layers represent foundational cell lineages that subsequently give rise to specific sub-lineages (e.g., the bone, connective tissue and circulatory system (mesoderm), the digestive tract (endoderm), and the epidermis and sensory-nervous system (ectoderm)). Following this initial step, cellular differentiation proceeds iteratively over the remainder of development, with increasingly specialized cell types emerging and forming complex, ordered structures including tissues and organs as a result of migration, cell-cell contacts and/or spatially-patterned lineage commitment decisions.

Terminal cellular differentiation, whereby a cell takes on a fixed phenotype without further scope for specialization, is often associated with growth arrest. The cessation of cell division may be conditional, for example, fibroblasts (i.e., mesodermal cells which make up the “bricks and mortar” of connective tissue) can be triggered to resume dividing in response to injury. In other situations, the capacity to divide is completely lost, as seems to be generally the case for mature neurons. Some tissues undergo continuous cell turnover over the lifetime of the organism, for example the blood, dermis, and intestinal epithelium. It has been discovered that in many, if not all, such cases these tissues are replenished from a small reservoir of multipotent “stem cells” which have the capacity both to self-renew and to give rise to a range of different cell types. For example, the diverse terminally-differentiated cell types of the blood (macrophages, neutrophils, natural killer cells, B and T lymphocytes, etc.) are replenished from a pool of self-renewing hematopoietic stem cells (“HSCs”) resident in the bone marrow via intermediate “progenitor” cells with more limited proliferative and lineage potential. Stem cell and intermediate progenitor populations have also been identified in other tissues such as the muscle and the lining of the gut. Generally, cellular differentiation in vivo is believed to be a “one-way street” in that during the expansion of any given clone of cells the members of the clone either maintain a constant phenotype or take on more specialized sub-lineages. It is possible that limited dedifferentiation can occur in situations such as wound healing, but claims that cells spontaneously “transdifferentiate” to completely different lineages in vivo (e.g., from fibroblast to neuron) or sometimes “regress” to become stem cells in response to stress have failed to gain wide acceptance.

Genetically identical cells within a single organismal cell community can stably take on very different gene expression profiles owing to the character of the genomic regulatory networks found in metazoans. The regulatory network can be conceptualized as a directed graph representing the influence of the transcriptional activity of each gene on the other genes (nodes) in the network. The periphery of the network, where most of the genes reside, comprises terminal nodes corresponding to genes encoding “effector” proteins that determine the broad phenotypic characteristics of the cell, including enzymes and structural proteins. The compact core of the network comprises genes that express regulatory proteins, including “transcription factors”, which interact in a sequence-specific manner with cis-acting regulatory regions in the DNA. Transcription factors influence the activity of target genes located near their cognate DNA binding sites, typically either by enhancing or blocking the recruitment of transcriptional machinery through the action of peptide transactivation domains (“TADs”). These factors control the expression of the peripheral effector genes in master-slave relationships, often co-regulating the expression of entire “gene batteries” (i.e., sets of functionally-related genes that act together under specific conditions or within specific cell types). Transcription factors can also regulate the activity of other transcription factors, either positively or negatively. Many examples of auto-regulating and cross-regulating transcription factors are found in metazoan genomic regulatory networks. These network relationships limit and define the stable patterns of gene expression accessible to the network and the transitions permitted between states. As an example, a common network motif features a pair of “master regulator” transcription factors that positively autoregulate while negatively cross-regulating each other, these two factors also controlling distinct effector gene batteries associated with alternative cell lineage fates. In this scenario, the two master transcription factors are both inactive early on in development. Subsequently, one or other factor is nudged into activity and locks itself and its associated effector gene battery into a stable “ON” state, while simultaneously suppressing the activity of the other factor and its downstream effector battery. These events at the genomic regulatory network level underpin a stable differentiation event at the level of cellular phenotype. The trigger pushing this “bistable” sub-net to commit might involve signal transduction (e.g., readout of a threshold level of one of the graded extracellular “morphogen” factors which establish spatial coordinate systems in the developing embryo). Alternatively, the sub-net might be evolutionarily tuned to generate divergent lineages probabilistically in the appropriate ratio as a consequence of gene expression “noise,” or some combination of cue-driven and stochastic commitment could be built into the architecture of the genetic network.

The understanding that cross-linkages within genomic regulatory networks constrain them to a limited number of “attractor” states out of an almost limitless number of potential expression profiles suggests the idea that the differentiation status of a cell could be profoundly influenced by artificially-induced changes in the levels of a small number of transcription factors. It has been shown that the ectopic expression of even a single master regulator factor in cultured cells can, in some cases, unleash a cascade of secondary gene expression changes and bring on a lineage switch. For example, a few days' sustained expression of the myogenic transcription factor MyoD from a transgene in fibroblasts is sufficient to convert many of the targeted cells into multinucleate, muscle-like cells bearing little resemblance to the starting fibroblasts. More commonly, the joint expression of multiple transcription factors (sometimes in conjunction with microRNAs) from transgenes has been required to drive “direct conversion” or “transdifferentiation” from one terminally-differentiated cell type to another at reasonable levels of efficiency. Examples include the conversion of fibroblasts into neurons using the transcription factor combination Ascl1, Brn2 and Myt1l or to macrophages using PU.1 and C/EBPa. It should be noted that while there is often a wholesale remodeling of cellular phenotype in these experiments, consistent with the “attractor” idea, it remains uncertain how fully these artificially-induced fate conversions recapitulate the results of normal development.

### Reprogramming to Pluripotency

Cellular differentiation has been analogized to the process of a ball rolling down an inclined landscape starting from a high point that corresponds to an entirely uncommitted state in the early embryo, and progressing through a branching landscape of valleys corresponding to the increasingly specialized lineage choices made during development. This “Waddington landscape” (named for biologist C. H. Waddington) can also be thought of as an “attractor landscape” at the level of the transcriptional networks governing cell phenotype. In the type of direct conversion described above, the forcing input of transgene expression allows the network to overcome an energy barrier and traverse from one of the valleys near the bottom of the hill to a neighboring valley. An even more dramatic overriding of the natural course of fate determination would be to push the ball from the bottom of the landscape (the terminally-differentiated state) all the way back up the hill to the embryonic state. The 2012 Nobel Prize in Medicine was awarded to two scientists, Sir John Gurdon and Shinya Yamanaka, who proved such a reversal, is in fact feasible. Gurdon's early work on cloning (somatic cell nuclear transfer) showed that the cytoplasm of an oocyte contains factors that can reset the nucleus of a differentiated cell back to an embryonic “ground state.” Half a century later, informed by new understanding of the role played by genomic regulatory networks in the specification of cell fate, Yamanaka searched for a combination of transcription factors whose joint expression would suffice to completely dedifferentiate a terminally-differentiated somatic cell. Yamanaka focused on transcription factors known to be particularly active in the embryonic stem cells (“ESCs”) which have been derived from the inner cell mass of the early embryo. These cells are known to be “pluripotent,” which is to say they can give rise to all three of the founding lineages which emerge at gastrulation and thus ultimately to all the tissues of the adult organism. Yamanaka used retroviral vectors to co-express diverse combinations of his candidate factors in mouse fibroblasts and screened the cultures for colonies bearing molecular markers of pluripotency. Using this approach, he was able to identify a combination or “cocktail” of four transcription factors whose co-expression is sufficient to reliably convert a small percentage of the targeted fibroblasts into ESC-like cells. The “Yamanaka factors”, as they became known, are Oct4, Sox2, Klf4 and c-Myc, and the cocktail is frequently referred to by the acronym “OSKM.” The cells produced using Yamanaka's approach are designated “induced pluripotent stem cells” or iPSCs. The term “cellular reprogramming” is commonly used to refer specifically to the derivation of iPSCs, although “reprogramming” is also sometimes used more broadly to describe artificially-induced lineage conversion in general.

Yamanaka's breakthrough inaugurated a burgeoning new field of biomedical research based on the derivation and application of iPSCs. These cells circumvent the ethical concerns that have limited the application of ESC and, unlike ESCs, they can readily be derived from parental cells of any genetic background desired, e.g., cells taken from patients with genetically-linked diseases. The iPSCs can theoretically be used to produce cells of any somatic lineage, and protocols based on specific culture conditions and cytokines exist for producing many cell types of interest, for example cardiomyocytes, T cells, and various neuronal sub-types. Ultimately, experts predict it may well be possible to use patient-specific iPSCs to make immunologically-compatible cells, tissues and organs for diverse applications in regenerative medicine.

A major stumbling block to the therapeutic application of iPSCs derived using Yamanaka's original retroviral transgene delivery system is that it leaves copies of powerful, potentially immortalizing transgenes scattered through the genomes of the reprogrammed cells. The development of safer approaches based on “non-integrating” or “footprint-free” expression vectors quickly became a priority for stem cell researchers. Of the numerous different technical approaches which have been described, the three which have found the most adherents are based on, respectively: (a) episomal DNA, (b), Sendai virus, and (c) mRNA transfection. Episomal vectors are circular DNA constructs similar to plasmids in that they are carried in the nucleus of target cells. They are distinguished from regular plasmids by the presence of a eukaryotic origin of replication which prevents the rapid dilution of the vector in dividing cell populations and gives a much greater perdurance of transgene expression. The Sendai virus has a completely cytoplasmic, RNA-based life cycle, in contrast to retrovirus and lentivirus which survive by inserting a copy of their genome into the host cell's nuclear genome. Messenger RNA is rapidly degraded in the cytoplasm after delivery to cells and is usually re-administered on a daily basis during the reprogramming process. Comparing the popular footprint-free systems, the episomal DNA and Sendai-based approaches offer the simplicity of “one-shot” transgene delivery, but entail the inconvenience of downstream cleanup and/or screening steps along with some residual risk that vector elements could persist in the wake of reprogramming. The mRNA system sidesteps these safety concerns and is thus the most clinically relevant of the three methods.

The drawbacks and limitations of the mRNA reprogramming system currently relate to the need for repeated delivery of the vector to the target cells. By using doxycycline-controlled expression of integrated lentiviral Yamanaka factors, researchers have shown that the OSKM combination needs to be expressed for weeks in human fibroblasts to fully activate the endogenous “pluripotency circuit” and lock in commitment to the pluripotent state. Early mRNA reprogramming protocols called for 14-18 transfections at 24-hour intervals in order to robustly generate iPSC colonies with useful efficiency. Thus, a substantial commitment of hands-on time is required (with no relief for weekends and holidays) and this has been a factor slowing the uptake of mRNA reprogramming compared to the episomal and Sendai techniques. A second important limitation of the mRNA system is that the need for repeated dosing makes the application of the method challenging in some cell types of interest. The first cells to be reprogrammed to pluripotency were fibroblasts, and this is still the most popular starting cell type for iPSC derivation. Fibroblasts are relatively easy to culture from skin biopsies and are among the most tractable and long-lived primary cells available for in vitro work. This has led to their popularity as a model system and the existence of many large patient-specific fibroblast banks. Fortunately, it is easy to achieve efficient mRNA transfection into fibroblasts and it has been found that their transfectability actually improves after expression of the Yamanaka factors pushes them to undergo mesenchymal-epithelial transition. A few other somatic cell types have been identified that might be preferred over fibroblasts as starting material for iPSC derivation in some settings, (e.g., because they can be obtained using less invasive techniques). These alternative cell types include: adipose-derived stem cells (“ADSCs”), which can be isolated from liposuction aspirates; keratinocytes, which can be cultured from the roots of plucked hairs; urine-derived renal epithelial cells, which are easily isolated and cultured from urine samples; blood-derived cells including true blood lineages (e.g., peripheral blood mononuclear cells and lymphocytes) and endothelial progenitors, which can be obtained from a regular blood draw or, in some cases, a finger prick. All of the aforementioned cell types can be reprogrammed using viral or episomal techniques, but so far the ease of mRNA reprogramming in fibroblasts has only been recapitulated in the urine-derived epithelial cells. In the case of blood cells, at least, it is clear that the difficulty of achieving sustained transgene expression from mRNA in these cells represents a major hurdle to implementing effective mRNA reprogramming protocols.

One strategy for simultaneously addressing the inconvenience of current mRNA protocols and opening up additional cell types to mRNA reprogramming involves potentiating the cocktail of reprogramming factors so that pluripotency can be induced with an abbreviated regimen of transfections. The scientific literature contains numerous reports of alternative reprogramming factors or factor combinations which, at least in certain contexts, lead to faster and more productive reprogramming. Two alternative reprogramming factors identified by James Thomson, Nanog and Lin28, both enhance reprogramming kinetics and productivity when used in conjunction with the four Yamanaka factors in the context of mRNA reprogramming. Engineered reprogramming factors have also been described that can accelerate the activation of the endogenous pluripotency circuit. In this approach, the activity of an established reprogramming factor is enhanced by expressing it as a fusion protein featuring one or more additional TADs. In some cases, TADs for the construction of such chimeric reprogramming factors have been isolated from proteins which are known to produce unusually strong transactivating effects, without any connection to the regulation of pluripotency. For example, TADs derived from MyoD transcription factor and from the viral transactivator VP16 have both been used to enhance the activity of Oct4 in reprogramming. In the setting of mRNA reprogramming, an enhanced, 6-factor derivative of Yamanaka's original OSKM cocktail featuring an Oct4-MyoD TAD fusion (M₃O) along with Nanog and Lin28 cuts the number of transfections needed for reprogramming by roughly 50% relative to the originally-presented OSKM and OSKM+Lin28 mRNA protocols. Using this potentiated cocktail, high iPSC productivity can generally be achieved with nine days of transfection, and a few colonies can often be obtained from as little as five or six transfections. This reduced time has made it possible to establish robust second-generation mRNA reprogramming protocols that avoid the need for a feeder cell layer, an important desideratum for clinical application.

Hundreds of different transcription factors have been identified in the genomes of animals, microorganisms and even viruses. In principle, transactivating domains isolated from any of these factors might enhance the speed and/or efficiency of cell lineage conversion when fused to known reprogramming factors. It is an empirical question which chimeric factors can offer a benefit within a given setting defined by cell type, reprogramming factor combination and stoichiometry, time course of ectopic gene expression, delivery vector employed, etc. The Gal4 transcription factor from yeast has been used for decades as a model system to develop our understanding of how genetic transcription is regulated, and the structure and function of this protein's component domains has been dissected and analyzed extensively in the scientific literature. Because Gal4 naturally occurs in a single-celled organism, its native role is far removed from the control of cellular differentiation, let alone the induction of pluripotency. Consequently, its use in a fusion construct is unique and the results obtained unexpected. The methods and compositions described herein pertain to the application of chimeric reprogramming factors featuring the C-terminal transactivating domain excerpted from the Gal4 in the context of mRNA-based lineage conversion.

Production of Synthetic mRNA

Methods for mass-producing long, single-stranded RNA (“ssRNA”) molecules are well known to those of skill in the art. While RNA oligomers up to a few dozen nucleotides in length can be made using chemistries similar to those employed to manufacture PCR primers, longer RNA molecules can currently only be mass-produced using enzymatic techniques. Single-stranded RNA molecules in the size range of hundreds to thousands of nucleotides with specific sequence composition can be generated in bulk in enzymatic reactions employing recombinant versions of phage RNA polymerase enzymes, including the T3, T7 and SP6 RNA polymerases. This general approach is referred to as in vitro transcription (“IVT”) and has been practiced by molecular biologists for decades. Various commercial kits are available that streamline and optimize the procedure, for example the MEGAscript kit (Thermo Fisher, Waltham, Mass.) and HiScribe kit (NEB, Ipswich, Mass.). In IVT reactions an RNA polymerase and a DNA template are added to a buffer containing ribonucleotide triphosphates. The DNA template contains the complementary sequence required to template transcription of the desired RNA positioned downstream of a short promoter region whose sequence is specific to the phage polymerase of choice. Only the promoter needs to be double-stranded, although in practice the template is usually a fully double-stranded PCR product or a cut plasmid. The RNA polymerase enzyme is highly processive and upon binding the promoter normally transcribes the template sequence into a single RNA transcript until it reaches the end of the DNA template, whereupon it is released to carry out further rounds of transcription. Transcription continues until the NTPs are depleted. Typically, IVT reactions are run for several hours and yield tens or hundreds of RNA molecules for every molecule of DNA template. The DNA template can be degraded away by addition of a recombinant DNase enzyme if desired. In most applications, it is necessary to purify the RNA product from the IVT buffer components, e.g., using traditional precipitation-based methods or the convenient spin columns available for this purpose such as those in the popular MEGAclear kit (Thermo Fisher, Waltham, Mass.).

Exogenous RNA engages innate immune antiviral defense pathways on delivery to mammalian cells in culture, and this can lead to deleterious consequences including suppressed translation of synthetic mRNA transcripts, release of stress-associated cytokines, cell apoptosis and senescence. These effects are dose dependent and tend to become more pronounced on repeat administration owing to sensitization of the cells mediated by the activation of Type I interferon signaling. Incorporation of certain modified nucleobases in synthetic mRNA transcripts (e.g., pseudouridine, 2-thiouridine, 5-methylcytidine and 5-methoxycytidine, can reduce the immunogenicity of the material). Several suitable modified nucleotides are available commercially and these can be incorporated into synthetic transcripts through partial or total substitution of the corresponding canonical form of the nucleotide in the IVT reaction buffer. In addition, sophisticated RNA purification methods such as the use of HPLC or size-exclusion columns can be applied to lower the residuum of immunogenic IVT side-products such as the short transcripts that are produced by abortive transcription events in these reactions.

Naturally-occurring mRNA molecules are long ssRNAs incorporating an Open Reading Frame (“ORF”) which encodes a polypeptide, this protein coding sequence being delimited by start and stop codons. Importantly, additional features must be present in order for the mRNA molecule to be efficiently translated in a cell. In eukaryotes, ribosomes are normally recruited to the 5′ end of the RNA by a “cap”, which is added to nascent RNA transcripts enzymatically in the nucleus. This structure comprises a guanosine nucleotide covalently bonded to the 5′ end of the transcript by a distinctive triphosphate bridge. Accessory proteins bind the cap and facilitate recruitment of the ribosome, which subsequently starts scanning down the RNA and initiates translation on reaching the first start codon (i.e., a 5′-AUG-3′ triplet). In order to be efficiently translated, mRNA must also incorporate a “polyA tail” at its 3′ end. The tail is a homopolymeric riboadenosine tract of tens to hundreds of bases length. As with the 5′ cap, the 3′ tail is added enzymatically to nascent message transcripts within the nucleus in eukaryotic cells. PolyA binding proteins (“PABPs”) bind the tail in the cytoplasm and these promote ribosome recruitment and recycling via looping interactions with protein complexes bound to the 5′ cap. It is known that translation of mRNA transcripts is much diminished in the absence of either the cap or the tail structures, and drastically curtailed when both features are absent. Enzymatic removal of the cap and tail is part of the normal cellular mRNA turnover pathway, effectively inactivating transcripts before they are fully degraded. The translational activity and functional lifetime of mRNA transcripts is also influenced by the content of untranslated regions (“UTRs”) flanking the protein coding region. The sequence content of the 5′ and 3′ UTRs and their functional impacts are highly diverse. It is known that the immediate sequence context of the start codon has an impact on the rate of translation, and preferred “Kozak sequences” that extend into the start-codon proximal bases of the 5′ UTR have been identified which promote efficient translational initiation. Otherwise, most of the sequence motifs that have been catalogued pertaining to the UTRs relate to conditional down-regulation (e.g., by presenting target sites for the binding of microRNAs expressed in specific developmental contexts).

In order to act effectively as mRNA on delivery to the cytoplasm of cells in vivo or in vitro, artificial ssRNAs made using IVT reactions should incorporate the key features of natural mRNA, including the 5′ cap and polyA tail structures. Methods for making synthetic mRNA with these features are known to those skilled in the art. The cap can be added enzymatically to transcripts after the IVT reaction is complete using a recombinant version of an RNA capping enzyme isolated from the Vaccinia virus. Kits for enzymatic capping are currently available from CELLSCRIPT (Madison, Wis.) and NEB (Ipswich, Mass.). The cap structure added by the viral enzyme closely resembles the native cap structure found in eukaryotic mRNA. An alternative approach is “co-transcriptional capping,” based on the inclusion of a synthetic “cap analog” in the IVT reaction buffer. This technique relies on the fact that the 5′ nucleotide in IVT transcripts is templated from the 3′ end of the phage polymerase promoter and is therefore fixed. In the case of T7 RNA polymerase, this base is always a ‘G,’ and a 5′ cap can be incorporated into a high percentage of transcripts by substituting a synthetic di-guanosine dinucleotide for a fraction of the rGTP in the reaction buffer. For example, when 80% of the rGTP normally included in an IVT reaction is replaced by such a cap analog, 80% of RNA transcripts can be expected to incorporate the cap structure at the 5′ end. Several cap analogs are commercially available, their chemical structures matching the natural cap with varying degrees of fidelity. Some of the low-cost analogs have the drawback that they are only incorporated into transcripts with the preferred stereochemistry 50% of the time, lowering the activity of the resulting mRNA inside the cell. Currently, “Anti-Reverse Cap Analog” (ARCA) is the cap analog of choice as it closely mimics the natural eukaryotic cap and is always incorporated with the appropriate stereochemistry. Novel cap analogs have been described with special features such as resistance to the decapping enzymes involved in mRNA turnover and might offer future performance benefits. Although convenient, co-transcriptional capping tends to be relatively expensive because IVT reaction yields fall sharply as the rGTP concentration is sacrificed to attain higher capped-product fractions. By contrast, enzymatic capping can in the best case achieve near-100% capping efficiency without entailing any compromise of IVT yields. As it is technically difficult to routinely assay the capped fraction achieved in practice, potential batch-to-batch variation in mRNA activity is of concern when using the enzymatic method. Given this balance of pros and cons, both the enzymatic and co-transcriptional capping strategies find adherents among those skilled in the art of making synthetic mRNA.

As with capping, the incorporation of the polyA tail can also be achieved either through an enzymatic post-IVT step or co-transcriptionally in the IVT reaction itself. Again, the two approaches have balanced advantages and disadvantages and both strategies are in widespread use. Commercially available tailing enzyme reagents can be used to add polyA tails of up to several hundred bases to IVT reaction products. Alternatively, co-transcriptional addition of the polyA tail can be driven through the use of an IVT template incorporating an oligo(dT) tract downstream of the 3′ UTR template. This approach simplifies the workflow and is more conducive to achieving a consistent product than enzymatic capping. It can be challenging to maintain plasmid constructs with homopolymeric runs as these features promote plasmid recombination and instability in bacterial culture. This is a hurdle to the application of co-transcriptional tailing when it is desired to use linearized plasmid directly as an IVT template. Some practitioners have addressed this issue through the use of low-recombination bacterial strains. Alternatively, the oligo(dT) stretch can be incorporated into PCR products generated by amplification of untailed plasmid sequences using heeled reverse primers. The PCR approach has the benefit that large quantities of IVT template can be made up from small, miniprep-scale plasmid stocks. There is a practical limit on the length of the oligo(dT) tract that can be introduced via the heeled primer approach owing to the size limits on primer synthesis. Experiments have shown that a polyA tail of around 30 nucleotides is the minimum size required to give strong translation. Increasing the length of the tail to 60-120 nucleotides gives markedly higher translational activity, but the improvements seem to taper off after that. Currently, the heeled primer technique can readily be applied to produce synthetic mRNA with polyA tails of 120 nucleotides length.

Whatever the preferred capping and tailing strategies employed, the foundation for a synthetic mRNA production pipeline is generally a DNA template construct featuring an RNA polymerase promoter, a 5′ UTR, a protein coding sequence and a 3′ UTR. While the UTR sequences used in such constructs could in principle be taken from the natural mRNAs encoding the protein to be expressed, a more typical practice is to employ an optimized and tested generic UTR framework for all such constructs. For example, some workers use a 5′ UTR incorporating an AT-rich, low-secondary structure leader adapted from the tobacco etch virus genome upstream of a strong Kozak consensus sequence along with a 3′ UTR sequence excerpted from one of the long-lived globin transcripts. The assembly of such constructs is a straightforward application of well-established molecular biology techniques for one skilled in the art. For example, standard oligo synthesis, PCR and cloning techniques can be used to create a plasmid vector containing the generic parts of the template, and the precisely delimited coding sequences for the proteins of interest can be PCR-amplified from a cDNA prep or an extant plasmid and cloned into this vector to produce complete, gene-specific mRNA synthesis templates. In recent years the generation of such constructs has been considerably simplified by the emergence of novel cloning approaches such as Gibson Assembly and various forms of Ligation-Independent Cloning. These techniques support efficient, seamless assembly of multiple DNA fragments without the need for the extraneous restriction sites required by traditional cloning methods. In addition, the rise of low-cost commercial “gene synthesis” services now makes it economically feasible to have large fragments or entire multi-kilobase DNA constructs made to order. The de novo gene synthesis approach facilitates implementation of constructs featuring “codon optimized” ORFs (to enhance translation kinetics and/or mRNA half-life) and engineered ORFs encoding, for example chimeric, fusion proteins with improved or novel functionality.

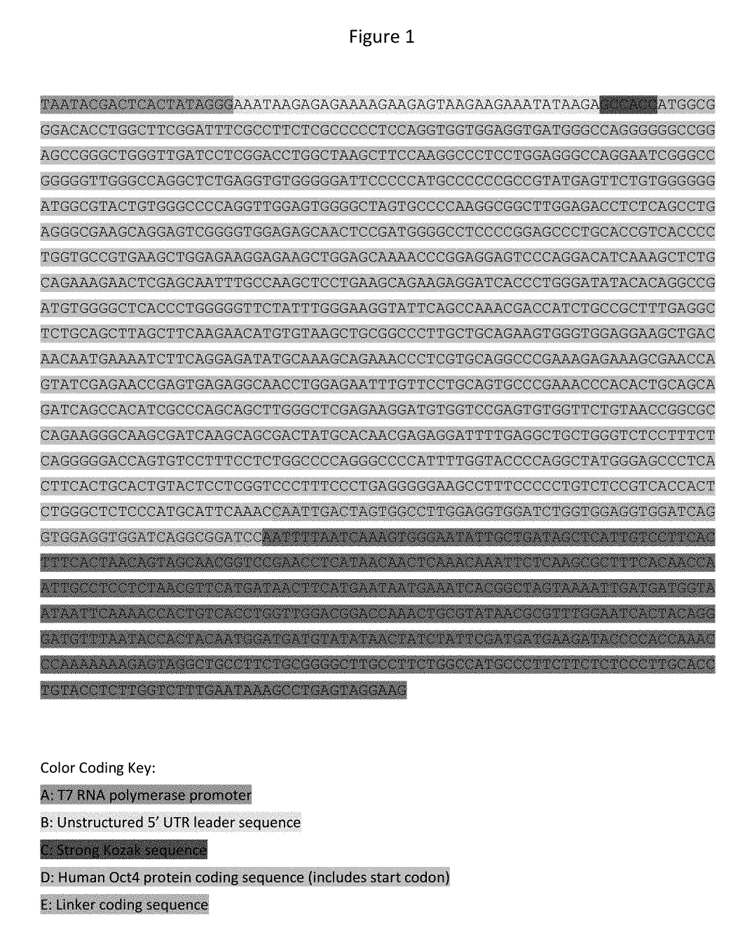

FIG. 1 (SEQ ID NO. 1) herein provides a sequence that can used to construct an IVT template for an Oct4-Gal4 TAD fusion construct. Other sequences relevant to the construction of IVT templates for Sox2 and its engineered Sy (Sox2-YAP TAD) variant, Klf4, c-Myc, Lin28 and Nanog may be obtained from Warren et al. (Scientific Reports 2:657, 2012), Warren et al. (Current Protocols in Stem Cell Biology, 4A.6.1-4A 6.27, 2013), Warren et al. (Cell Stem Cell 7(5):618-30, 2010), from international patent application PCT/US2016/069079 and from U.S. Pat. No. 8,802,438.

Delivery of mRNA into Cultured Cells

The delivery of synthetic mRNA into cultured cells can be achieved using the same basic methods applied to deliver other nucleic acids such as plasmids and siRNAs. There are two common approaches: (a) chemical transfection, and (b) electroporation. In the chemical transfection approach the RNA is complexed with a cationic (i.e., positively-charged) “vehicle” and then added to cell culture media. The positive charges drive ionic bonding of the vehicle to the negatively-charged nucleic acid, forming molecular complexes or “nanoparticles” on the order of tens of nanometers in diameter. The presence of cationic chemical groups on the vehicle and the overall charge neutralization resulting from complexation facilitates the accretion of the RNA-containing nanoparticles to the negatively-charged plasma membrane of cells. The vehicle typically features a lipid or polymer backbone whose lipophilic character also contributes to the attachment of complexes to the cell membrane. The plasma membrane of mammalian cells turns over gradually as patches of membrane sporadically invaginate, encapsulating membrane-bound material, and bud off as vesicles called “endosomes” inside the cell. This natural process of “endocytosis” brings surface-bound RNA/vehicle complexes into the cell. The fate of internalized vesicles varies depending on the specific endocytic pathway involved, but the spontaneous release of intact endosomal contents into the cytoplasm is generally disfavored. The manufacturers of transfection reagents have developed chemical strategies to promote endosomal escape (e.g., by exploiting the low pH characteristic of endosomal compartments). Nonetheless, while it can be expected that a significant fraction of complexed mRNA delivered to culture media binds to cells and is eventually internalized, typically only a small fraction of that material will be released productively to the cytoplasm. In spite of this bottleneck there are a number of cationic transfection reagents on the market which can deliver physiologically useful titers of synthetic mRNA into cell types of interest, including RNAiMAX, MessengerMAX, and Lipofectamine 2000 (Life Technologies, San Diego, Calif.), Stemfect (Stemgent, Lexington, Mass.), Trans-IT mRNA (Mirus Bio, Madison, Wis.) and mRNA-In (GlobalStem, Gaithersburg, Md.). The cytotoxicity of these reagents is generally quite low and in some cases the same reagent can be used to transfect short single-stranded or double-stranded RNA (e.g., siRNA or miRNA). The transfection process itself is generally very simple: synthetic mRNA is mixed with vehicle at an empirically-determined optimum ratio in a buffer solution, incubated for a few minutes and then either pipetted onto cell cultures or diluted into bulk culture media immediately before performing media changes. The efficiency of transfection is sensitive to culture media formulation and tends to vary with cell density (often becoming poor at high confluence), all of which can present challenges to protocol optimization. However, the major limitation with these reagents is the low penetrance of transfection achievable in some important cell types of interest, notably the blood lineages. This is especially problematic when using mRNA as an expression vector as each transfection gives only a transient burst of protein expression owing to mRNA turnover and cell division. Transcription factors are generally short-lived proteins and daily mRNA delivery is typically needed to sustain their robust expression in lineage conversion applications. When well under 50% of cells take up significant amounts of RNA, as is typical going into blood cells with cationic reagents, the percentage of cells that experience prolonged, uninterrupted factor expression on repeat transfection is inevitably very small. The other main approach to mRNA transfection mentioned above, electroporation, offers a way around this difficulty. In this technique target cells are resuspended in a buffer containing mRNA and subjected to a pulsed electric field. The pulses create short-lived rips or holes in the plasma membrane, permitting RNA to enter the cytoplasm by passive diffusion before the membrane heals. This technique is most readily applied to suspension cells since adherent cells (e.g., fibroblasts) typically have to be detached and brought into suspension before the electroporation procedure is performed. Electroporation can deliver mRNA efficiently to blood cells given appropriate optimization of experimental parameters such as the electric pulse waveform and the buffer concentration of mRNA. Unfortunately, electroporation is a relatively harsh procedure, and a prolonged regimen of daily electroporation is unlikely to be well tolerated by target cells.

The twin hurdles presented by low-penetrance delivery using cationic reagents and the high cytotoxicity associated with electroporation put a premium on abbreviating the mRNA dosing schedule required to effect lineage conversion in blood cells. This need is addressed by the compositions and methods described herein.

The mRNA cocktail used to induce pluripotency should include transcripts encoding at least four reprogramming factors from the group Oct4, Sox2, Klf4, Lin28, Nanog and Myc (either c-Myc or L-Myc), and transcripts representing at least one factor from the group Oct4, Sox2 and Nanog should be present in the form of a Gal4 TAD chimera. Aside from the Gal4 TAD, other engineered enhancements over the wild-type version of the reprogramming factors may also be represented within the cocktail, e.g., Sy can be substituted for Sox2, and where applicable these attributes can be combined with Gal4 chimerism in the same factor. The individual transcripts encoding the selected factors should be present at from 5% to 50% by mass of the mRNA cocktail. The preferred combination and stoichiometry of factors will vary according to the target cell type, and can be optimized straightforwardly by scoring a matrix of alternative cocktail recipes in reprogramming trials based on the yield of TRA-1-60⁺ colonies at the end of the run. In general, a good starting point is to include all the selected factors in equimolar ratio (based on the computed molecular weight of each transcript species). The most important reprogramming factor, Oct4, and any engineered factors present in the mix should be prioritized as variables in stoichiometry optimization. The mRNA cocktail should be delivered to cells at 24-hours intervals for from 3 to 5 days. When using cationic transfection reagents to deliver the mRNA, a suitable daily dose range to evaluate for fibroblasts is from 100 to 1000 ng per well in 6-well format. Dosing can easily be optimized for specific conditions (such as the cell type and transfection reagent) based on a dose-ramp reprogramming trials.

### Delivery of Reprogrammed Cells into the Patient

Reprogrammed cells may be introduced into a patient by injection or by surgical methods known to those skilled in the art. Transfected cells that have been reprogrammed may be introduced into the patient at or near the location desired. This may be a site where cells naturally exist of a type that match the newly reprogrammed cell type or they may be injected at a location containing cells of a different cell type. The reprogrammed cells may be re-introduced into the patient from whom they were extracted or into different patient. Further, the patient may be human or non-human and the reprogrammed cells may also be introduced into a different species.

### EXAMPLES

Described herein is one exemplary method of reprogramming human fibroblasts based on delivery of a 6-factor synthetic mRNA cocktail that includes a transcript encoding an Oct4-Gal4 TAD fusion protein. Remarkably, this method robustly and efficiently makes iPSCs from low-passage human fibroblasts in a feeder-free setting with as few as four or five transfections, less than a third of the number required by the first working mRNA reprogramming protocol described by Warren et al. (Cell Stem Cell 7(5):618-630, 2010).

### Example 1

### Ultra-Rapid mRNA Reprogramming of Fibroblasts using an Oct4-Gal4 TAD Fusion Construct

### A. IVT Templates

The IVT templates for making individual components of the mRNA cocktail are produced by PCR amplification of miniprepped plasmid constructs. The individual constructs can be produced by cloning DNA fragments representing the coding sequence for each protein of interest into a generic plasmid host vector featuring a T7 promoter, low-secondary structure 5′ UTR with a strong Kozak sequence, a 3′ UTR excerpted from the murine alpha-globin transcript, and a T7 terminator. The coding sequence inserts can be de novo synthesized DNA fragments made using, for example, the gBlocks service offered by Integrated DNA Technologies (“IDT”) (Coralville, Iowa). The vector plasmid can also be made-to-order, e.g., using IDT's MiniGene synthesis service. These fragments can be seamlessly cloned into the vector at the junction of the 5′ and 3′ UTR sequences using, for example, the HiFi DNA Assembly Cloning Kit (NEB, Ipswich, Mass.). To generate large quantities of linear IVT template DNA featuring oligo(dT) runs to template co-transcriptional addition of a 120-nucleotide polyA tail, a construct clone should be PCR amplified using a high-fidelity DNA polymerase (e.g., using HiFi Hotstart Master Mix (Kapa Biosystems, Wilmington, Mass.)) with a forward primer that binds upstream of the T7 promoter and a PAGE-purified, T₁₂₀-heeled reverse primer with a binding site that precisely abuts the end of the 3′ UTR. PCR products should be column-purified before being taken forward to IVT reactions.

Six templates are required for the protocol described below, containing the coding sequences for the Oct4-Gal4 TAD fusion protein, the Sox2-YAP TAD fusion protein (Sy) described in the references, wild-type Klf4, the T58A mutant form of c-Myc (often used in reprogramming because of its heightened potency relative to wild-type c-Myc), Lin28 and Nanog.

### B. IVT Reactions

IVT reactions are performed at the 40 μl scale using the MEGAscript kit (Thermo Fisher, Waltham, Mass.). Approximately 0.5-1 μg of DNA template should be used per reaction at this reaction scale. The standard riboNTPs included in the MEGAscript kit should be replaced by a blend of ARCA cap analog and rATP, 5-methoxy-CTP, rGTP, and rUTP. ARCA cap analog and 5-methoxy-CTP are available from Trilink Biotechnologies (San Diego, Calif.). A 4:1 ratio of ARCA to rGTP is used to ensure the production of a high percentage of capped RNA (nominally, 80% at this ARCA:rGTP ratio). Assembled reactions should be incubated for 4 hours at 37° C. and subjected to a 15-minute TURBO DNase treatment to digest the template as per the Ambion manual. The reaction is purified using MEGAclear columns (Thermo Fisher, Waltham, Mass.), and treated with Antarctic Phosphatase (NEB, Ipswich, Mass.) to remove immunogenic 5′ triphosphate moieties from the uncapped RNA fraction. The RNA should be re-purified on spin columns and quantitated (e.g., using a UV spectrophotometer). The individual factors should be diluted with pH 7.0 TE (Tris-EDTA) buffer to make 100 ng/μl working stocks.

### C. Cocktail Assembly

The 100 ng/μl working stocks of the individual mRNAs should be combined in approximately equimolar ratio to make up a 100 ng/μl working stock of reprogramming cocktail, as follows:

| | |
|---|---|
| Oct4-Gal4 TAD Fusion | 18% |
| Sy | 21% |
| Klf4 | 19% |
| c-Myc T58A | 18% |
| Lin28 | 10% |
| Nanog | 14% |

D. Transfection of mRNA

The desired amount of RNA should be diluted along with mRNA-In transfection reagent (GlobalStem, Rockville, Md.) at a ratio of 5 μL of reagent per microgram of mRNA in calcium-and magnesium-free DPBS at a final mRNA concentration of 10 ng/μl. The transfection cocktails should be incubated for 10 minutes and then added to reprogramming media at a final RNA concentration of 200 ng/ml. Whenever RNA is delivered in reprogramming media, the media should also be supplemented with B18R interferon inhibitor (eBioscience, San Diego, Calif.) at a final concentration of 100 ng/ml. The media should be used promptly after supplementation with mRNA to perform a media change on the cells under reprogramming.

### E. Reprogramming Media

The reprogramming media is E6 (Thermo, Carlsbad, Calif.) supplemented with Lipid Mixture and Poloxamer 188 (Sigma, St. Louis, Mo.), human platelet lysate (Biological Industries, Cromwell, Conn.), and 20 ng/ml bFGF (basic Fibroblast Growth Factor).

### F. Plating of Fibroblasts

Set multiple reprogramming wells with human fibroblasts at different densities as line-to-line variation in growth characteristics will lead to significant variation in the optimal starting cell density for reprogramming. When working with vigorous, low-passage fibroblasts in a 6-well format it generally works well to set cultures with 30K, 60K and 90K cells. It can be helpful to increase these numbers if working with slow-growing fibroblasts, or decrease them if working with highly robust, proliferative cells. Pre-coat culture wells with recombinant Laminin 521 (BioLamina, Sundbyberg, Sweden) per the manufacturer's instructions. Plate fibroblasts in FibroGRO Xeno-Free Fibroblast Expansion Media (EMD Millipore, Billerica, Mass.) the day before the first transfection. Cells should be cultured at 5% O₂ tension as this strongly enhances the efficacy of mRNA reprogramming. Media should be pre-equilibrated in the CO₂/O₂-regulated incubator for 1-4 hours before being applied to cells.

### G. Reprogramming Regimen

Deliver mRNA cocktails to cells by media change as described above, starting on the day after plating and repeating four more times at 24-hour intervals. Note that the first transfection media change defines the start of “day 0” in the protocol timeline. For the best results, tailor the dosage of mRNA-supplemented media to the density of the reprogramming culture, e.g., use 1 ml media when cells are sparse, 1.5 ml when cells reach medium density, and 2 ml or more at near or full confluence.

### H. Emergence of Colonies

From day 5 on, replace media daily using regular Nutristem XF pluripotent stem cell expansion media (Biological Industries, Cromwell, Conn.). Immature “pre-colonies” will normally be apparent by the end of the reprogramming phase and mature-looking colonies with classic iPSC morphology should be observed starting around day 6 or day 7. Colonies can be picked or alternatively bulk-passaged en masse using EDTA to establish “passage 1” (P1) iPSC cultures for expansion and stabilization. Oct4/TRA-1-60 costaining of fixed and permeabilized reprogramming cultures or expansion cultures can be used to confirm the presence of characteristic human pluripotent stem cell markers.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

I claim:

1. A method for accelerating cell lineage conversion comprising the steps of transfecting a cell with a composition comprising at least one synthetic mRNA encoding a engineered chimeric transcription factor incorporating a heterologous peptide sequence derived from the C-terminal transactivation domain (TAD) of Gal4, wherein the activity of said chimeric transcription factor is enhanced by the presence of said fused transactivation domain thereby promoting accelerated lineage conversion as compared to other methods of cell lineage conversion.

2. The method according to claim 1, wherein said cell lineage conversion is a dedifferentiation, a transdifferentiation or a directed differentiation.

3. The method according to claim 2, wherein said cell is a somatic cell.

4. The method according to claim 3, wherein said cell lineage conversion reprograms said somatic cell into an induced pluripotent stem cell.

5. The method according to claim 3, wherein said somatic cell is selected from the group consisting of fibroblasts, renal epithelial cells, keratinocytes, adipose-derived stem cells, mesenchymal stem cells, blood-derived endothelial progenitors and peripheral blood mononuclear cells.

6. The method according to claim 1, wherein said engineered chimeric transcription factor(s) are based on Oct4 and/or Sox2 and/or Nanog.

7. The method according to claim 6, wherein said composition comprises synthetic mRNAs encoding wild-type, mutant or engineered forms of at least four factors from the group Oct4, Sox2, Klf4, Lin28, Nanog and Myc (either c-Myc or L-Myc).

8. The method according to claim 1, wherein said cell is a human cell.

9. The method according to claim 1, wherein said cell is a non-human cell.

10. A method of cell therapy comprising:
isolating somatic cells from a patient;
transfecting said somatic cells with a composition comprising at least one mRNA encoding one or more chimeric transcription factors having a heterologous peptide sequence derived from the C-terminal transactivation domain (TAD) of Gal4, wherein the activity of said chimeric transcription factor is enhanced by the presence of said transactivation domain;
and administering said transfected cells into said patient.

11. The method of claim 10, wherein said somatic cells are genetically modified prior to the step of administering said transfected cells into said patient.

12. The method of claim 10, wherein said transfecting of said somatic cells reprograms said somatic cells to pluripotency.

13. The method of claim 12, wherein said pluripotent cells are differentiated in vitro before the step of administering said cells into said patient.
